(19) United States
(12) Patent Application Publication
(10) Pub. No.: US 2002/0142284 A1
(43) Pub. Date: Oct. 3, 2002

(54) Methods of identifying renal protective factors

(76) Inventors: Debasish Raha, New Haven, CT (US); Cyndi D. Green, Madison, CT (US); Richard L. Cate, Weston, MA (US)

Correspondence Address: MINTZ, LEVIN, COHN, FERRIS,, GLOVSKY AND POPEO, P.C., One Financial Center, Boston, MA, 02111, US

(21) Appl. No.: 09/905,325

(22) Filed: Jul. 13, 2001

Related U.S. Application Data
(60) Provisional application No. 60/217,932, filed on Jul. 13, 2000.

(51) Int. Cl. C12Q 1/00; C12Q 1/68

(52) U.S. Cl. 435/4; 435/6

(57) ABSTRACT

Disclosed are methods of identifying toxic agents, e.g., renal toxic agents, using differential gene expression. Also disclosed are novel nucleic acid sequences whose expression is differentially regulated by renal injury agents.

This application claims priority to U.S. Pat. No. b 60/217,932, filed July 13, 2000 which is incorporated herein by reference in its entirety

### FIELD OF THE INVENTION

The invention relates generally to the identification of renal protective agents in kidney tissue using differential gene expression.

### BACKGROUND OF THE INVENTION

The primary function of the mammalian kidney is to filter waste products and excess fluid from the blood; in humans, the two kidneys of an adult filter about 200 quarts of fluid daily. In addition to filtering wastes from the blood, the kidneys also release hormones that regulate blood pressure, control the production of red blood cells, and synthesize essential vitamins. Pathologies associated with impaired or altered kidney function affect hundreds of thousands of adults and children annually in the United States, and include ischemic kidney injury, renal transplantation, drug toxicity, cancer, diabetes, hypertension, glomerulonephritis, childhood lupus nephritis, and polycystic kidney disease. Treatments and therapies which prevent or ameliorate the onset and/or progression of renal disease are of vital importance.

### SUMMARY OF THE INVENTION

The invention is based in part on the discovery that certain nucleic acids are differentially expressed in renal tissue of animals subjected to repeated ischemic injury compared with animals subjected to a single acute ischemic injury. These differentially expressed nucleic acids include novel sequences and nucleic acid sequences that, while previously described, have not heretofore been identified as renal injury responsive.

In various aspects, the invention includes methods of screening a test agent for toxicity, e.g., renal toxicity. For example, in one aspect, the invention provides a method of identifying a renal toxic agent by providing a test cell population comprising a cell capable of expressing one or more nucleic acid sequences responsive to renal injury, contacting the test cell population with the test agent and comparing the expression of the nucleic acid sequences in the test cell population to the expression of the nucleic acid sequences in a reference cell population not treated with a renal injury. An alteration in expression of the nucleic acid sequences in the test cell population compared to the expression of the gene in the reference cell population indicates that the test agent is renal toxic.

In an another aspect, the invention provides a method of assessing the renal toxicity of a test agent in a subject. The method includes providing from the subject a cell population comprising a cell capable of expressing one or more renal injury responsive genes, and comparing the expression of the nucleic acid sequences to the expression of the nucleic acid sequences in a reference cell population that includes cells from a subject whose exposure status to a renal toxic agent is known. An alteration in expression of the nucleic acid sequences in the test cell population compared to the expression of the nucleic acid sequences in the reference cell population indicates the renal toxicity of the test agent in the subject.

In a further aspect, the invention provides a method of screening a test agent with renal injury activity. For example, in one aspect, the invention provides a method of identifying a renal injury agent by providing a test cell population comprising a cell capable of expressing one or more nucleic acid sequences responsive to renal injury, contacting the test cell population with the test agent and comparing the expression of the nucleic acid sequences in the test cell population to the expression of the nucleic acid sequences in a reference cell population not subjected to a renal injury. An alteration in expression of the nucleic acid sequences in the test cell population compared to the expression of the gene in the reference cell population indicates that the test agent is a renal injury modulator, e.g. a renal protective factor (RPF).

Also provided are novel nucleic acids, as well as their encoded polypeptides, whose expression is responsive to the effects of renal injury.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention is based in part on the discovery of changes in expression patterns of multiple nucleic acid sequences in rodent kidney cells following exposure to renal injury agents. By comparing the genes differentially expressed in response to repeated ischemic injury, it is possible to generate gene profiles capable of distinguishing between renal protective and renal injury genes expressed in renal tissue.

The renal injury includes venous inclusion for 30 minutes and bilateral ureteral obstruction at various days prior to tissue collections. Kidneys were harvested from mice surgically treated under one of several protocols: 8 days after 30 minute venous occlusion causing bilateral ischemia; 15 days after bilateral ischemia; or 8 days after bilateral ureteral obstruction. cDNA was prepared and the resulting samples were processed through using GENECALLING™ differential expression analysis as described in U. S. Pat. No. 5,871,697 and in Shimkets et al., Nature Biotechnology 17:798-803 (1999). The contents of these patents and publications are incorporated herein by reference in their entirety. A summary of the treatment conditions and data analysis parameters is shown in Table 1.

Genes that either protect renal tissue from ischemic injury or participate in renal damage were identified by measuring MRNA that were either up-or down-regulated in animals subjected to repeated ischemic events induced for example by 30 minute venous occlusions as compared to animals subjected to a single ischemic event induced by a 30 minute venous occlusion. 104 single copy nucleic acid sequences were identified whose expression levels differed in treated renal tissue. These sequences are referred to herein as RPF 1-104. A summary of the RPF sequences analyzed is presented in Tables 2-4.

Genes that were coordinately differentially expressed, e.g. downregulated or upregulated, 8 days after 30 minute venous occlusion causing bilateral ischemia; 15 days after bilateral ischemia; and 8 days after bilateral ureteral obstruction are listed in Table 2. Theses genes are responsible for protection of renal function.

Two sequences (RPF 1-2) represent novel mouse genes. The other 102 sequences identified have been previously described (RPF 3-104). For some of the novel sequences (i.e., a cloned sequence is provided. Also provided is a consensus sequences which includes a composite sequence assembled from the cloned and additional fragments. For a given RPF sequence, its expression can be measured using any of the associated nucleic acid sequence in the methods described herein. For previously described sequences (RPF 3-104), database accession numbers are provided. This information allows for one of ordinary skill in the art to deduce information necessary for detecting and measuring expression of the RPF nucleic acid sequences.

TABLE 1

| COMPARISON | COMPARISON OF |
|---|---|
| CODES | TREATMENTS |
| 1 | A vs. C |
| 2 | F vs. I |
| 3 | P vs. M |
| 4 | B vs. D |
| 5 | E vs. H |
| 6 | K vs. J |
| 7 | K vs J |
| 8 | B vs. A (not inspected) |
| 9 | D vs. C (not inspected) |
| 10 | E vs. F (not inspected) |
| 11 | H vs. I (not inspected) |

| Treatment | Treatment | Harvest |
|---|---|---|
| identifier | type | Day |
| A | Ischemia, 30 minutes on day 0 | 15 |
| B | Ischemia, 30 minutes on day 0; 30 | 15 |
| | minutes on day 15 | |
| C | Sham surgery, day 0 | 15 |
| D | Sham surgery, day 0; ischemia, 30 | 17 |
| | minutes, day 15 | |
| E | Ischemia, 30 minutes, day 0; | 10 |
| | ischemia, 30 minutes, day 8 | |
| F | Ischemia, 30 minutes, day 0 | 8 |
| H | Sham surgery, day 0; ischemia, 30 | 10 |
| | minutes, day 8 | |
| I | Sham surgery, day 0 | 8 |
| J | Sham surgery, day 0 | 2 |
| K | Ischemia, 30 minutes, day 0 | 2 |
| M | Sham surgery, day 0 | 8 |
| P | Bilateral ureteral obstruction, day 0 | 8 |

TABLE 2

| Upregulated genes | Acc. No. | RPF Assignment |
|---|---|---|
| Lipocalin | 147696 | 3 |
| Osteopontin | j04806 | 4 |
| Clusterin | D140775 | |
| Lysozyme M | m21047 | 6 |
| Astrocytic phospho- | g49234 | 7 |
| protein PEA-1 | | |
| ICAM-1 | m90551 | 8 |
| Annexin II | m14044 | 9 |
| Thrombospondin | J05605 | 10 |
| SPARC | x04017 | 11 |
| Carboxypeptidase E | u23184 | 12 |
| Peptidyl-proline | x67809 | 13 |
| isomerase C | | |
| C1 inhibitor | af010254 | 14 |
| γ-Actin | m21495 | 15 |
| Apolipoprotein E | M12414 | 16 |
| MHC class II I-A beta | v01527 | 17 |
| TIMP-2 | x62622 | 18 |
| Adseverin | u04354 | 103 |
| Virus-like retroposon | x17124 | 104 |

| Up-regulated ESTs | | RPF Assignment |
|---|---|---|
| aa023491 | | 19 |
| aa689813 | | 20 |
| aa466100 | | 21 |
| ai27242 | 22 | |
| aa986962 | | 23 |

| Down-regulated genes | Acc. No. | RPF Assignment |
|---|---|---|
| Acyl CoA dehydrogenase | u07159 | 24 |
| Cytochrome P450 4B1 | d50834 | 25 |
| α-Methylacyl CoA | u89906 | 26 |
| dehydrogenase | | |
| UDP glucuronyl-S-transferase | d87866 | 27 |
| γ-Glutamyltranspeptidase | u30509 | 28 |
| Down-regulated ESTs | | RPF Assignment |
| ai530049 | | 29 |
| ai006567 | | 30 |
| ai647790 | | 31 |
| aa261635 | | 32 |

TABLE 3
COMPARISON OF TREATMENT

| | | | COMPARISON CODE | | | | | |
|---|---|---|---|---|---|---|---|---|
| Gene Name | Accno | RPF# | 1 | 2 | 3 | 4 | 5 | 6 |
| Novel Fragment: [C] [N] | cgmms0t0386.2_12773- | 1 | — | 2.1 | — | — | — | 1.7 |
| | 312 | | | | | | | |
| Novel Fragment: [C] [N] | cgml0y0318.5_12773-13 | 2 | — | 2.5 | — | — | — | — |
| LCN2: Mus musculus | 147696 | 3 | 4.4 | 6.3 | 10.6 | −3.6 | −1.9 | 66.2 |
| neutrophil gelatinase- | | | | | | | | |
| associated lipocalin | | | | | | | | |
| precursor | | | | | | | | |
| Osteopontin: Mus | j04806 | 4 | 3.8 | 4.9 | 3.4 | — | −1.6 | 12 |
| musculus osteopontin | | | | | | | | |
| mRNA, complete cds. | | | | | | | | |
| CLU or MSGP-2 or APOJ: | d14077 | 5 | 4 | 5.1 | 6.8 | — | — | 10.4 |
| Mouse clusterin (aka | | | | | | | | |
| sulfated glycoprotein-2, | | | | | | | | |
| apolipoprotein J) | | | | | | | | |
| LYZ: Mouse lysozyme | m21047 | 6 | 13.7 | 1.5 | 3.7 | 2.7 | 3.9 | 2.4 |
| gene. | | | | | | | | |
| PEA15: Mouse | g49234 | 7 | 2.9 | 4.7 | 2.8 | −2.9 | — | 7.3 |
| astrocytic | | | | | | | | |
| phosphoprotein PEA-15 | | | | | | | | |
| ICAM1: Mouse | m90551 | 8 | 5 | 9.8 | 2 | 1.5 | — | −1.9 |
| intercellular adhesion | | | | | | | | |
| molecule 1 | | | | | | | | |
| CAL1H or ANX2: Mouse | m14044 | 9 | 2.5 | 3.2 | 2 | −1.8 | −1.1 | 6.2 |
| annexin II (aka calpactin I | | | | | | | | |
| heavy chain) | | | | | | | | |
| THBS1 OR TSP1: | cgmmi0q0432.7_2773- | 10 | 3.5 | 4.5 | 2.8 | — | — | 5.1 |
| Mouse thrombospondin | 221 J05605 | | | | | | | |
| (THBS1) gene | | | | | | | | |
| SPARC: Mouse mRNA | x04017 | 11 | 4.5 | 2 | 1.6 | 1.7 | −1.5 | 3.4 |
| for cysteine-rich | | | | | | | | |
| glycoprotein SPARC. | | | | | | | | |
| CPE: Mus musculus | u23184 | 12 | 2 | 2.7 | 4.6 | — | 1.5 | 2.7 |
| musculus | | | | | | | | |
| carboxypeptidase E | | | | | | | | |
| (Cpe) mRNA, complete | | | | | | | | |
| cds. | | | | | | | | |
| CYPC or PPIC *: | x67809 | 13 | 2.2 | 2.6 | 3.3 | 2.7 | −3.7 | 1.9 |
| M.musculus mama | | | | | | | | |
| mRNA. | | | | | | | | |
| C1 inhibitor: Mus | af010254 | 14 | 1.6 | 2.6 | 1.5 | 2.3 | 2 | 2.6 |
| musculus C1 inhibitor | | | | | | | | |
| mRNA, complete cds. | | | | | | | | |
| ACTG1 or ACTG: | m21495 | 15 | 1.7 | 2.1 | 1.8 | −2 | 1.8 | 1.8 |
| Mouse cytoskeletal actin | | | | | | | | |
| 2 (gamma-actin) | | | | | | | | |
| APOE: Mouse | m12414 | 16 | 3.2 | 3.2 | 5.6 | 2.6 | 3 | −1.5 |
| apolipoprotein E mRNA | | | | | | | | |
| [C] | | | | | | | | |
| H2-IABETA: Mouse H-2 | v01527 | 17 | 5.3 | 3.3 | 2 | 3.2 | — | — |
| class II histocompatibility | | | | | | | | |
| antigen, A beta chain | | | | | | | | |
| TIMP2: M.musculus | x62622 | 18 | 2 | 3.6 | 3.2 | 1.6 | — | 1.6 |
| TIMP-2 mRNA for tissue | | | | | | | | |
| inhibitor of | | | | | | | | |
| metalloproteinases. | | | | | | | | |
| ABP1 OR DAO1 OR | gbem_aa023491 | 19 | 9.2 | 7.4 | 2.8 | — | 2.1 | −3.2 |
| AOC1: mh74e11.r1 | | | | | | | | |
| Soares mouse placenta | | | | | | | | |
| 4NbMP13.5 14.5 Mus | | | | | | | | |
| musculus cDNA; mouse | | | | | | | | |
| homolog of diamine | | | | | | | | |
| oxidase, aka amiloride | | | | | | | | |
| binding protein, | | | | | | | | |
| histaminase | | | | | | | | |
| CAL1H or ANX2: Mouse | gbem_aa689813 | 20 | 2.4 | 3.8 | 1.9 | −1.8 | −1.1 | 6.3 |
| calpactin I heavy chain | | | | | | | | |
| (aka annexin II) | | | | | | | | |
| RIC: same as ion | gbem_aa466100 | 21 | 1.8 | 2.9 | 1.8 | — | — | 2.5 |
| channel homolog | | | | | | | | |
| RIC or EF8: Mouse ion | gbem_ai272427 | 22 | 3.2 | 3.7 | 2 | — | — | 4 |
| channel homolog RIC | | | | | | | | |
| (aka EF-8) | | | | | | | | |
| ACTGCS: Mouse | gbem_aa986962 | 23 | 1.9 | 2.1 | 1.9 | — | 1.8 | 1.9 |
| gamma actin | | | | | | | | |
| MCAD: Mus musculus | u07159 | 24 | −1.9 | −2.6 | −1.8 | — | — | −2 |
| medium-chain acyl-CoA | | | | | | | | |
| dehydrogenase (Acadm) | | | | | | | | |
| mRNA, complete cds. | | | | | | | | |
| (map:3 76.3cM) EC | | | | | | | | |
| 1.3.99.3 | | | | | | | | |
| CYP4B1: Mus musculus | d50834 | 25 | −2.8 | −2.5 | −4.2 | −1.7 | 2 | −5.7 |
| cytochrome P450 4B1 | | | | | | | | |
| u89906: Mus musculus | u89906 | 26 | −2.3 | −2.9 | −3.8 | — | −1.9 | −4.6 |
| alpha-methylacyl-CoA | | | | | | | | |
| racemase mRNA, | | | | | | | | |
| complete cds. | | | | | | | | |
| UGT1: Mouse UDP- | d87866 | 27 | — | 4.5 | 17.7 | — | 7.3 | 2.1 |
| glucuronosyltransferase | | | | | | | | |
| 1—1 microsomal | | | | | | | | |
| GGT: Mus musculus | u30509 | 28 | −2 | −2.2 | −2.1 | 1.6 | 5.7 | −2.9 |
| gamma- | | | | | | | | |
| glutamyltranspeptidase | | | | | | | | |
| gbem ai530049: | gbem_ai530049 | 29 | −3.5 | −3.7 | −6.6 | −2.2 | — | −19.9 |
| ui88f01.y1 Sugano | | | | | | | | |
| mouse liver mila Mus | | | | | | | | |
| musculus cDNA clone | | | | | | | | |
| [C] | | | | | | | | |
| gbem ai006567: | gbem_ai006567 | 30 | −3.8 | −5.3 | −2.6 | — | — | −10.2 |
| ue14d08.y1 Sugano | | | | | | | | |
| mouse embryo mewa | | | | | | | | |
| Mus musculus cDNA | | | | | | | | |
| clone 1480335 | | | | | | | | |
| gbem ai647790: | gbem_ai647790 | 31 | −3.7 | −3.2 | −2 | — | — | −5.6 |
| uk43b06.x1 Sugano | | | | | | | | |
| mouse kidney mkia Mus | | | | | | | | |
| musculus cDNA clone | | | | | | | | |
| [N] | | | | | | | | |
| gbem aa261635: | gbem_aa261635 | 32 | −2.1 | −2.5 | −2.8 | 1.9 | — | −6.6 |
| mz87d12.r1 Soares | | | | | | | | |
| mouse NML Mus | | | | | | | | |
| musculus cDNA clone | | | | | | | | |
| 720407 5′ | | | | | | | | |
| cgmmu0w0167 12783- | cgmmu0w0167_12783- | 33 | −2.7 | −2.3 | −7.7 | — | — | −5.9 |
| 50: | 50 | | | | | | | |
| cgmmw0n0249.6 12783- | cgmmw0n0249.6_12783- | 34 | −3.3 | −4.3 | −14.6 | — | −1.8 | −18 |
| 8: | 8 | | | | | | | |
| C3: Mouse complement | j00369 | 35 | −2 | 1.5 | 1.6 | 1.7 | −2.2 | −3.1 |
| component C3 gene, 3′ | | | | | | | | |
| end. | | | | | | | | |
| IGH: Mouse germline | j00434 | 36 | 1.8 | 3.5 | 1.9 | 1.7 | −1.7 | −2.5 |
| IgH chain gene, DJC | | | | | | | | |
| region: segment D- | | | | | | | | |
| FL16.1. | | | | | | | | |
| VCAM1: Mus musculus | u12878 | 37 | 4.6 | 4.2 | 1.9 | 3.6 | 1.8 | −3.2 |
| NIH Swiss vascular cell | | | | | | | | |
| adhesion molecule-1 | | | | | | | | |
| (VCAM-1) | | | | | | | | |
| ARG2: Mus musculus | af032466 | 38 | 1.7 | 1.9 | 5 | −1.5 | — | −2.2 |
| arginase II mRNA, | | | | | | | | |
| complete cds. | | | | | | | | |
| CLU OR MSGP-2 OR | cgmmb1i0346_2776-394 | 39 | — | 2.7 | 4 | — | −2.4 | 7.9 |
| APOJ: Mouse mRNA for | | | | | | | | |
| sulfated glycoprotein-2; | | | | | | | | |
| aka SGP-2, CLUSTRIN, | | | | | | | | |
| APOLIPOPROTEIN J | | | | | | | | |
| (APO-J) [C] | | | | | | | | |
| CYP24: Mus musculus | cgmmk0n0430.7_12783- | 40 | 1.3 | — | — | −3.1 | −6.1 | 3.2 |
| mRNA for vitamin D-24- | 192 | | | | | | | |
| hydroxylase D89669; | | | | | | | | |
| CYTOCHROME P450- | | | | | | | | |
| CC24, | | | | | | | | |
| MITOCHONDRIAL | | | | | | | | |
| [Precursor] | | | | | | | | |
| DAN or DANA or NBL1: | d50263 | 41 | 1.9 | 1.7 | 1.6 | 1.9 | 1.9 | 1.7 |
| Mus musculus zinc | | | | | | | | |
| finger protein DAN | | | | | | | | |
| ADAMTS1: Mouse | d67076 | 42 | −1.6 | 2.4 | 3.5 | — | 1.8 | 1.7 |
| disintegrin and | | | | | | | | |
| metalloproteinase | | | | | | | | |
| containing | | | | | | | | |
| thrombospondin motifs | | | | | | | | |
| KRT8 or KRT2-8: Mouse | d90360 | 43 | 1.8 | 1.9 | 2.2 | — | — | 2.7 |
| gene for cytokeratin endo | | | | | | | | |
| A. | | | | | | | | |
| TSC22 or TFB1l4: | gbem_aa023495 | 44 | 1.6 | 2.6 | 1.7 | — | −1.7 | 3.9 |
| Mouse putative | | | | | | | | |
| regulatory protein TSC- | | | | | | | | |
| 22 (aka TGFB-stimulated | | | | | | | | |
| clone 22) | | | | | | | | |
| mt35q06.r1: Mouse | gbem_aa186086 | 45 | 4 | 4.2 | 2.6 | 1.8 | — | 1.5 |
| mt35q06.r1 Soares | | | | | | | | |
| mouse 3NbMS Mus | | | | | | | | |
| musculus, cDNA clone | | | | | | | | |
| IMAGE: 623098 5′, | | | | | | | | |
| mRNA sequence - Mus | | | | | | | | |
| musculus. 446 bp [N] | | | | | | | | |
| ve81e09.r1: ve81e09.r1 | gbem_aa423762 | 46 | 1.9 | 2.5 | 1.9 | — | — | 2.8 |
| Soares mouse mammary | | | | | | | | |
| gland NbMMG Mus | | | | | | | | |
| musculus cDNA clone | | | | | | | | |
| [N] | | | | | | | | |
| gbem aa462111: | gbem_aa462111 | 47 | −1.8 | −1.9 | −1.5 | — | — | −7.1 |
| vg72f02.r1 Soares | | | | | | | | |
| mouse NbMH Mus | | | | | | | | |
| musculus cDNA clone | | | | | | | | |
| 871515 5′ similar to | | | | | | | | |
| TR: G164423 G164423 | | | | | | | | |
| SUCCINYL- | | | | | | | | |
| COA: ALPHA-KETOACID | | | | | | | | |
| COENZYME A | | | | | | | | |
| TRANSFERASE | | | | | | | | |
| PRECURSOR | | | | | | | | |
| PRECURSOR | | | | | | | | |
| HSP27 or HSP25: | gbem_aa592259 | 48 | — | 4.6 | 1.7 | — | — | 3 |
| Mouse heat shock 27 | | | | | | | | |
| kDa protein (aka HSP25) | | | | | | | | |
| gbem aa689673: | gbem_aa689673 | 49 | — | 2.8 | 2.3 | — | — | 3.9 |
| vs03b08.r1 Barstead | | | | | | | | |
| mouse irradiated colon | | | | | | | | |
| MPLRB7 Mus musculus | | | | | | | | |
| cDNA [N] | | | | | | | | |
| APOE: Mouse | gbem_ai048801 | 50 | 5.2 | — | 3.7 | 3.8 | 5.2 | −2.1 |
| apolipoprotein E | | | | | | | | |
| gbem ai314702: | gbem_ai314702 | 51 | −1.6 | −1.9 | −2.3 | — | −1.7 | −3.4 |
| ui27g01.x1 Sugano | | | | | | | | |
| mouse kidney mkia Mus | | | | | | | | |
| musculus cDNA clone | | | | | | | | |
| TROP2: mj67e12.y1 | gbem_ai595479 | 52 | — | 2.9 | 3.2 | −1.7 | 1.6 | 4.6 |
| Soares mouse | | | | | | | | |
| p3NMF19.5 Mus | | | | | | | | |
| musculus cDNA clone | | | | | | | | |
| [C] | | | | | | | | |
| TMSB4: Mouse | gbem_d76695 | 53 | 1.4 | 2.2 | 2.5 | — | — | 1.8 |
| thymosin beta-4 [C] | | | | | | | | |
| 102914: Mus musculus | 102914 | 54 | −1.6 | −1.7 | — | 1.7 | 3 | −3.3 |
| aquaporin-CHIP (aka | | | | | | | | |
| water channel protein for | | | | | | | | |
| red blood cells and | | | | | | | | |
| kidney proximal tubule, | | | | | | | | |
| early response protein | | | | | | | | |
| DER2) | | | | | | | | |
| F2R or PAR1 or CF2R: | 103529 | 55 | — | 2.2 | 2.3 | — | — | 3.8 |
| Mus musculus thrombin | | | | | | | | |
| receptor | | | | | | | | |
| FN1: Mouse fibronectin | m18194 | 56 | 4.9 | 4.2 | 1.8 | — | — | 2.9 |
| GLUT1 or SLC2A1: | m22998 | 57 | 1.8 | 2.1 | 2.2 | — | — | 2.2 |
| Mouse glucose | | | | | | | | |
| transporter type 1 | | | | | | | | |
| H2-D: Mus musculus H- | m34962 | 58 | 1.9 | 1.9 | 1.6 | 2.6 | — | — |
| 2 class I | | | | | | | | |
| histocompatibility | | | | | | | | |
| antigen, D—D alpha chain | | | | | | | | |
| (aka H2-D) | | | | | | | | |
| TlS21: Mouse NGF- | m64292 | 59 | 1.6 | 2.9 | — | — | −1.6 | 2.5 |
| inducible protein TlS21 | | | | | | | | |
| (aka BTG2) | | | | | | | | |
| JUNB: Mus musculus | u20735 | 60 | 2.5 | 4 | −2.7 | −2.3 | −2.8 | 2.7 |
| transcription factor junB | | | | | | | | |
| TAGLN or SM22 or | u36588 | 61 | — | 2.7 | 2.8 | — | 1.6 | 7.5 |
| SM22A: Mus musculus | | | | | | | | |
| smooth muscle protein | | | | | | | | |
| 22-alpha (aka transgelin, | | | | | | | | |
| actin-associated protein | | | | | | | | |
| p27 | | | | | | | | |
| BCL6: Mus musculus B- | uemm_1744_0 | 62 | 1.7 | 2.4 | 3 | 2.1 | — | 2.5 |
| cell lymphoma 6 protein | | | | | | | | |
| homolog | | | | | | | | |
| MMVL30: Mouse virus- | uemm_31615_0 | 63 | 2.4 | 3.8 | 2.9 | −2.6 | −1.6 | 18.7 |
| like (VL30) | | | | | | | | |
| retrotransposon BVL-1; | | | | | | | | |
| 1755 bp EST assembly | | | | | | | | |
| [N] | | | | | | | | |
| APOE: Mouse | uemm_3_0 | 64 | −2.4 | 4.7 | 3.7 | 3.8 | 5.2 | −2.9 |
| apolipoprotein E gene | | | | | | | | |
| [C] | | | | | | | | |
| C3: Mouse complement | uemm_463_0 | 65 | −2 | 5.8 | 8.1 | 1.7 | 3.5 | −3.1 |
| component C3 | | | | | | | | |
| CAL1L or S100A10: | uemm_474_0 | 66 | 1.6 | 2.7 | 1.8 | −2.9 | — | 4.2 |
| Mouse calpactin I light | | | | | | | | |
| chain (aka p11, p10 | | | | | | | | |
| protein, cellular ligand of | | | | | | | | |
| annexin II) | | | | | | | | |
| uemm 6447 0: | uemm_6447_0 | 67 | — | 1.7 | — | −1.5 | — | — |
| vb62b05.r1 Mus | | | | | | | | |
| musculus cDNA, 5″ end | | | | | | | | |
| [N] | | | | | | | | |
| TUBB5: Mouse tubulin | x04663 | 68 | 2.3 | 3.4 | −1.7 | 2.4 | −1.6 | 2.9 |
| beta-5 chain | | | | | | | | |
| CD14: Mouse CD14 | x13333 | 69 | 3.1 | 6.7 | 2.3 | −1.7 | −1.7 | 9.7 |
| mRNA for myelid cell- | | | | | | | | |
| specific leucine-rich | | | | | | | | |
| glycoprotein. | | | | | | | | |
| SGNE1: Murine | x15830 | 70 | — | 1.7 | 3.4 | — | — | 1.9 |
| neuroendocrine protein | | | | | | | | |
| 7B2 | | | | | | | | |
| KDAP: Mouse kidney- | d88899 | 71 | −1.6 | — | — | 3.8 | 2.1 | −5 |
| derived aspartic | | | | | | | | |
| protease-like protein | | | | | | | | |
| S100A11 or S100C: | gbem_aa003364 | 72 | 2 | 2.4 | 1.8 | — | — | 3.2 |
| Mouse endothelial | | | | | | | | |
| monocyte-activating | | | | | | | | |
| polypeptide (aka | | | | | | | | |
| calgizzarin, S100 | | | | | | | | |
| calcium-binding protein | | | | | | | | |
| A11) | | | | | | | | |
| PKM2: Mouse pyruvate | gbem_aa041815 | 73 | 1.6 | 1.7 | 2.7 | — | — | 1.9 |
| kinase, M2 isozyme | | | | | | | | |
| BGN: Mouse | 120276 | 74 | 2.5 | 3.3 | 1.9 | — | 1 | 1.9 |
| bone/cartilage | | | | | | | | |
| proteoglycan I (aka | | | | | | | | |
| biglycan, PG-S1) | | | | | | | | |
| PLAU: Mouse urokinas- | x02389 | 75 | — | 2.8 | 1.6 | 1.6 | 1.6 | — |
| type plasminogen | | | | | | | | |
| activator | | | | | | | | |
| x06358: Mouse mRNA | x06358 | 76 | −2.2 | −2.2 | −4.2 | −2.2 | −2.4 | −5.6 |
| for UDP- | | | | | | | | |
| glucuronosyltransferase | | | | | | | | |
| (EC 2.4.1.17). | | | | | | | | |
| CCNA2 or CCNA *: | x75483 | 77 | — | — | — | −2.2 | −2 | 2.5 |
| M.musculus G2/mitotic- | | | | | | | | |
| specific cyclin A2 | | | | | | | | |
| BGN *: Mouse | 120276 | 78 | 2.5 | 3.3 | 1.9 | — | 1 | 1.9 |
| bone/cartilage | | | | | | | | |
| proteoglycan I (aka | | | | | | | | |
| biglycan, PG-S1) | | | | | | | | |
| x06358 *: Mouse mRNA | x06358 | 79 | −2.2 | −2.2 | −4.2 | −2.2 | −2.4 | −5.6 |
| for UDP- | | | | | | | | |
| glucuronosyltransferase | | | | | | | | |
| (EC 2.4.1.17). | | | | | | | | |
| SLP2-c Mouse mRNA for | AB057756.1 | 80 | 5.8 | 4.3 | — | — | 1.9 | 3 |
| syrnaptotagmin-like | | | | | | | | |
| protein 2-c | | | | | | | | |

TABLE 4
COMPARISON OF TREATMENT

COMPARISON CODE

| GeneName | Accno | RPF# | 7 | 8 | 9 | 10 | 11 |
|---|---|---|---|---|---|---|---|
| cgmmu0w0167 12783-50: | cgmmu0w0167_12783- | 81 | −5.9 | — | — | — | — |
| | 50 | | | | | | |
| cgmmw0n0249.6 12783-8: | cgmmw0n0249.6_12783- | 82 | −18 | — | — | — | — |
| | 8 | | | | | | |
| CYP24: Mus musculus mRNA for | cgmmk0n0430.7_12783- | 83 | 3.2 | — | — | — | — |
| vitamin D-24-hydroxylase D89669; | 192 | | | | | | |
| CYTOCHROME P450-CC24, | | | | | | | |
| MITOCHONDRIAL [Precursor] | | | | | | | |
| CYP4B1: Mus musculus | d50834 | 84 | −5.7 | −1.9 | −3.3 | 3.7 | −3 |
| cvtochrome P450 4B1 | | | | | | | |
| gbem aa261635: mz87d12.r1 | gbem_aa261635 | 85 | −6.6 | — | −3.5 | −1.8 | −3.6 |
| Soares mouse NML Mus musculus | | | | | | | |
| cDNA clone 720407 5′ | | | | | | | |
| gbem aa462111: vg72f02.r1 | gbem_aa462111 | 86 | −7.1 | — | −3.1 | — | −2.9 |
| Soares mouse NbMH Mus | | | | | | | |
| musculus cDNA clone 871515 5′ | | | | | | | |
| similar to TR: G164423 G164423 | | | | | | | |
| SUCCINYL-COA: ALPHA- | | | | | | | |
| KETOACID COENZYME A | | | | | | | |
| TRANSFERASE PRECURSOR | | | | | | | |
| PRECURSOR | | | | | | | |
| gbem ai006567: ue14d08.y1 | gbem_ai006567 | 87 | −10.2 | −2.4 | −5 | −1.8 | −5.1 |
| Sugano mouse embryo mewa Mus | | | | | | | |
| musculus cDNA clone 1480335 | | | | | | | |
| gbem ai314702: uj27g01.x1 | gbem_ai314702 | 88 | −3.4 | — | — | — | — |
| Sugano mouse kidney mkia Mus | | | | | | | |
| musculus cDNA clone | | | | | | | |
| gbem ai530049: ui88f01.y1 | gbem_ai530049 | 89 | −19.9 | — | — | — | — |
| Sugano mouse liver mlia Mus | | | | | | | |
| musculus cDNA clone [C] | | | | | | | |
| gbem ai647790: uk43b06.x1 | gbem_ai647790 | 90 | −5.6 | — | −3.1 | — | −3.8 |
| Sugano mouse kidney mkia Mus | | | | | | | |
| musculus cDNA clone [N] | | | | | | | |
| 102914: Mus musculus aquaporin- | 102914 | 91 | −3.3 | 3.3 | −2.7 | 1.6 | −2.9 |
| CHIP (aka water channel protein | | | | | | | |
| for red blood cells and kidney | | | | | | | |
| proximal tubule, early response | | | | | | | |
| protein DER2) | | | | | | | |
| GGT: Mus musculus gamma- | u30509 | 92 | −2.9 | −1.7 | −2.2 | 3.6 | −2.5 |
| glutamyltranspeptidase | | | | | | | |
| u89906: Mus musculus alpha- | u89906 | 93 | −4.6 | — | −2.4 | — | −2.2 |
| methylacyl-CoA racemase mRNA, | | | | | | | |
| complete cds. | | | | | | | |
| CCNA2 or CCNA: M.musculus | x75483 | 94 | 2.5 | — | 4.4 | — | 3.6 |
| G2/mitotic-specific cyclin A2 | | | | | | | |
| KDAP: Mouse kidney-derived | d88899 | 95 | −5 | — | −2.3 | — | −3.6 |
| aspartic protease-like protein | | | | | | | |
| 102914 *: Mus musculus | 102914 | 96 | −3.3 | 3.3 | −2.7 | 1.6 | −2.9 |
| aquaporin-CHIP (aka water | | | | | | | |
| channel protein for red blood cells | | | | | | | |
| and kidney proximal tubule, early | | | | | | | |
| response protein DER2) | | | | | | | |
| GGT *: Mus musculus gamma- | u30509 | 97 | −2.9 | −1.7 | −2.2 | 3.6 | −2.5 |
| glutamyltranspeptidase | | | | | | | |
| u89906 *: Mus musculus alpha- | u89906 | 98 | −4.6 | — | −2.4 | — | −2.2 |
| methylacyl-CoA racemase mRNA, | | | | | | | |
| complete cds. | | | | | | | |
| x06358: Mouse mRNA for UDP- | x06358 | 99 | −5.6 | −1.7 | −3.4 | −2.5 | −2.4 |
| glucuronosyltransferase (EC | | | | | | | |
| 2.4.1.17). | | | | | | | |
| CCNA2 or CCNA *: M.musculus | x75483 | 100 | 2.5 | — | 4.4 | — | 3.6 |
| G2/mitotic-specific cyclin A2 | | | | | | | |
| 102914 *: Mus musculus | 102914 | 101 | −3.3 | 3.3 | −2.7 | 1.6 | −2.9 |
| aquaporin-CHIP (aka water | | | | | | | |
| channel protein for red blood cells | | | | | | | |
| and kidney proximal tubule, early | | | | | | | |
| response protein DER2) | | | | | | | |
| GGT *: Mus musculus gamma- | u30509 | 102 | −2.9 | −1.7 | −2.2 | 3.6 | −2.5 |
| glutamyltranspeptidase | | | | | | | |

Below follows additional discussion of nucleic acid sequences whose expression is differentially regulated after renal injury.

### RPF1

RPF1 is a novel 386 bp gene fragment. The nucleic acid has the following sequence:

| | |
|---|---|
| TCGCGTTCTCAATATTGGCATGAACCTGCTGATAAGCCATGTTGAGGAACAGGTATCTTTCCGACCTCC | (SEQ ID NO. 1) |
| TCATTGGTAAGCAGAGGCTGTAGGCTACGTGAACAACTGCAAAGAAGAAGCTCAGCAATCCCAGCTGT | |
| TTCCTGCACTGCAGCCAAGTATCCAGCCACGGGGGAAATCGGCGGTACTTAGTGCCATAATAAAGCTG | |
| ATACGCAGCTGCCAGGAGGCCAGCCAGGTACACCAGAGACAGCAGGGTGATGGCGACGATCGGACAAG | |
| GTTTTGTTCACAATCTCAATGGGAATCTTGTAAAAGTCACTCTGCTGGTTTCTGGCATATGGATGTATC | |
| ACATCTCTGACAAAGGAATAAAGAAAGAAAAATGTGGCCAAGCTT | |

A RPF 1 nucleic acids encodes a RPF 1 encodes a RPF polypeptide that includes the following amino acids sequence.

| | |
|---|---|
| ANEINAHVQQYAMNLFLYRESRRMPLCLSYAVHVVAFFFSLLGLQKRCQLWTDLWPPFRRYKTGYYLQYAAALLGALY | (SEQ ID NO. 2) |
| VLSLLTIAVIPLTKNVIEIPIKYFDSQQNRAYPHIVDRVFSLFFFTAL | |

### RPF2

RPF 2 is a novel 318 gene fragment. The nucleic acid has the following sequence:

| | |
|---|---|
| TCCGGAGGGTTAGCCCTGCATTCTGAGTTGGGAATATTGTCTTCCACGCCCCGGGAATCTCTGAATTTT | (SEQ ID NO. 3) |
| AGGAATTGTTTTGGGGAAGTAGCTTTTCTTCCCTTCTATTTAAATCCCGACTTTAAAGTTTAAAATCTCA | |
| AACTGTGAATTCCTAGAACTTCATTCTAAGCCGGACAATGTCAGCCATCAGTTGAGTTTGGCAGCAGTT | |
| AATTTCTATTTAACAAAATTTCTTATGGCCATCTGAGACCCCGGGACAGATCATAACTTCGAAACAGTT | |
| GGAACGAAACTCCAGAGACCGTGACTTCTAAATCCACTAGT | |

### General Methods

Several of the herein disclosed methods relate to comparing the levels of expression of one or more RPF nucleic acids in a test and reference cell populations. The sequence information disclosed herein, coupled with nucleic acid detection methods known in the are, allow for detection and comparasion of the various RPF transcripts. In some embodiments, the RPF nucleic acids and polupeptide correspond to nucleic acids or polypeptides which include the various sequences (referenced by RPF assignment numbers) disclosed for each RPF nucleic acid sequences.

In its various aspects and embodiments, the invention includes providing a test cell population which includes at least one cell that is capable of expressing one or more of the sequences RPF 1 - 1 04. By “capable of expressing” is meant that the gene is present in an intact form in the cell and can be expressed. Expression of one, some, or all of the RPFsequences is then detected, if present, and, preferably, measured. Using sequence information provided by the database entries for the known sequences, or the sequence information for the newly described sequences, expression of the RPF sequences can be detected (if present) and measured using techniques well known to one of ordinary skill in the art. For example, sequences within the sequence database entries corresponding to RPF sequences, or within the sequences disclosed herein, can be used to construct probes for detecting RPF RNA sequences in, e.g., northern blot hybridization analyses or methods which specifically, and, preferably, quantitatively amplify specific nucleic acid sequences. As another example, the sequences can be used to construct primers for specifically amplifying the RPFsequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

Expression can be also measured at the protein level, i.e., by measuring the levels of polypeptides encoded by the gene products described herein. Such methods are well known in the art and include, e.g., immunoassays based on antibodies to proteins encoded by the genes.

Expression level of one or more of the RPF sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING™ methods as described in U.S. Pat. No. 5,871,697 and in Shimkets et al., Nat. Biotechnol. 17:798-803.

In various embodiments, the expression of one or more sequences encoding genes of RPF 1-104 expressed in distinct gene profiles based on specific renal injury, as listed in Table 1, is compared. These gene profiles include, e.g., “up-regulated following repeated ischemic renal injury” (such as, e.g. RPF3-23 and 103-104) and “down-regulated following repeated ischemic renal injury” (such as, e.g. RPF 23-32). In some embodiments, expression of members of two or more gene profiles are compared.

In various embodiments, the expression of 2, 3, 4, 5, 6, 7,8, 9, 10, 15, 20, 25, 35, 40, 50, 100, or all of the sequences represented by RPF 1-104 are measured. Preferably, the expression of RPF 3-32 and 103-104 are measured. If desired, expression of these sequences can be measured along with other sequences whose expression is known to be altered according to one of the herein described parameters or conditions.

The reference cell population includes one or more cells for which the compared parameter is known. The compared parameter can be, e.g., renal toxic agent expression status or renal injury agent expression status. By “renal toxic agent expression status” is meant that it is known whether the reference cell has had contact with one or more renal toxic agents. Examples of renal toxic agents are, e.g., viruses, bacteria, drugs, transplantation, auto-immune antibodies, vasodilators, and vasoconstrictors. By “renal injury agent expression status” is meant that it is known whether the reference cell has had contact with a renal injury agent. Examples of renal injury agents include, e.g. ischemia, viruses, bacteria, drugs, transplantation, auto-immune antibodies, vasodilators, and vasoconstrictors. Whether or not comparison of the gene expression profile in the test cell population to the reference cell population reveals the presence, or degree, of the measured parameter depends on the composition of the reference cell population. For example, if the reference cell population is composed of cells that have not been treated with a known renal toxic agent, a similar gene expression level in the test cell population and a reference cell population indicates the test agent is not a renal toxic agent. Conversely, if the reference cell population is made up of cells that have been treated with a renal toxic agent, a similar gene expression profile between the test cell population and the reference cell population indicates the test agent is a renal toxic agent. As another example, if the reference cell population is composed of cells that have not been treated with a known renal injury agent, a similar gene expression level in the test cell population and a reference cell population indicates the test agent is not a renal injury agent. Conversely, if the reference cell population is made up of cells that have been treated with a renal injury agent, a similar gene expression profile between the test cell population and the reference cell population indicates the test agent is a renal injury agent.

In various embodiments, a RPF sequence in a test cell population is considered comparable in expression level to the expression level of the RPF sequence if its expression level varies within a factor of 2.0, 1.5, or 1.0 fold to the level of the RPF transcript in the reference cell population. In various embodiments, a RPF sequence in a test cell population can be considered altered in levels of expression if its expression level varies from the reference cell population by more than 1.0, 1.5, 2.0 or more fold from the expression level of the corresponding RPF sequence in the reference cell population.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations.

In some embodiments, the test cell population is compared to multiple reference cell populations. Each of the multiple reference populations may differ in the known parameter.

Thus, a test cell population may be compared to a first reference cell population known to have been exposed to a renal toxic agent, as well as a second reference population known to have not been exposed to a renal toxic agent.

The test cell population that is exposed to, i.e., contacted with, the test agent, e.g. renal toxic agent, can be any number of cells, i.e., one or more cells, and can be provided in vitro, in vivo, or ex vivo.

In other embodiments, the test cell population can be divided into two or more subpopulations. The subpopulations can be created by dividing the first population of cells to create as identical a subpopulation as possible. This will be suitable, in, for example, in vitro or ex vivo screening methods. In some embodiments, various sub populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell, e.g., kidney tissue. Kidney tissue includes mesangial cells, endothelial cells, glomerular cells, renal epithelial cells, embryonic kidney cells, or renal tubular cells. In other embodiments, the control cell is derived from a tumor. In some embodiments, the control cell is derived from the same subject as the test cell, e.g., from a region proximal to the region of origin of the test cell. In other embodiments, the reference cell population is derived from a plurality of cells. For example, the reference cell population can be a database of expression patterns from previously tested cells for which one of the herein-described parameters or conditions (e.g., renal toxic agent expression status) is known.

The test agent can be a compound not previously described or can be a previously known compound but which is not known to be a renal toxic agent or a renal injury modulating agent.

By “renal toxicity” is meant that the agent is damaging or destructive to kidney when administered to a subject that leads to kidney damage.

By “renal injury agent” is meant that the agent modulates (i.e., increases or decreases) renal injury. These agents include for example, ischemia, drugs, viruses, bacteria, vasoactive compounds, carbohydrates and polypeptides.

The subject is preferably a mammal. The mammal can be, e.g., a human, non-human 20 primate, mouse, rat, dog, cat, horse, or cow.

In humans, compounds provided as therapeutics may have effects specific to an individual. Differences in the genetic makeup of individuals can result in differences in their relative abilities to metabolize various drugs. An agent that is metabolized in a subject to act as a RPF agent can manifest itself by inducing a change in gene expression pattern from that characteristic of a pathophysiologic state to a gene expression pattern characteristic of a non-pathophysiologic state. Accordingly, the differentially expressed RPF sequences disclosed herein allow for a putative therapeutic or prophylactic agent to be tested in a test cell population from a selected subject in order to determine if the agent is a suitable RPF ligand in the subject.

Screening for Renal Toxic Agents

In one aspect, the invention provides a method of identifying toxic agents, e.g., renal toxic agents. The renal toxic agent can be identified by providing a cell population that includes cells capable of expressing one or more nucleic acid sequences homologous to those listed in Tables 2-4 as RPF 1-104. Preferably, the cell population includes cells capable of expressing one or more nucleic acid sequences homologous to RPF 1-104. The sequences need not be identical to sequences including RPF 1-104, as long as the sequence is sufficiently similar that specific hybridization can be detected. Preferably, the cell includes sequences that are identical, or nearly identical to those identifying the RPF nucleic acids shown in Tables 2-4. Preferably, RPF 1-2 and 33-102 are measured.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g., a sequence database, which assembles information about expression levels of known sequences following administration of various agents.

An alteration in expression of the nucleic acid sequence in the test cell population compared to the expression of the nucleic acid sequence in the reference cell population that has not been exposed to the test agent indicates the test agent is a renal toxic agent. For example, an alteration in expression of RPF 1-104 in the test cell population compared to the expression of the RPF 1-104 in the reference cell population that has not been exposed to the test agent indicates the test agent is a renal protective agent.

The invention also includes a renal toxic agent identified according to this screening method.

Assesing Toxicity of an Agent in a Subject

The differentially expressed RPF sequences identified herein also allow for the renal toxicity of a renal toxic agent to be determined or monitored. In this method, a test cell population from a subject is exposed to a test agent, i.e. a. renal toxic agent. If desired, test cell populations can be taken from the subject at various time points before, during, or after exposure to the test agent. Expression of one or more of the RPF sequences, e.g., RPF 1-104, in the cell population is then measured and compared to a reference cell population which includes cells whose renal toxic agent expression status is known. Preferably, the reference cells have not been exposed to the test agent. Preferably, RPF 1-2 and 33-102 are measured.

If the reference cell population contains no cells exposed to the treatment, a similarity in expression between RPFsequences in the test cell population and the reference cell population indicates that the treatment is non-renal toxic. However, a difference in expression between RPFsequences in the test population and this reference cell population indicates the treatment is renal toxic.

Screening for Renal Protective Agents

In one aspect, the invention provides a method of identifying renal protective agents. The renal protective agent can be identified by providing a cell population that includes cells capable of expressing one or more nucleic acid sequences homologous to those listed in Table 1 as RPF 1-104. Preferably, the cell population includes cells capable of expressing one or more nucleic acid sequences homologous to RPF 1-104. The sequences need not be identical to sequences including RPF 1-104, as long as the sequence is sufficiently similar that specific hybridization can be detected. Preferably, the cell includes sequences that are identical, or nearly identical to those identifying the RPF nucleic acids shown in Tables 2-4.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent, such as an ischemia-inducing compound or process.

An alteration in expression of the nucleic acid sequence in the test cell population compared to the expression of the nucleic acid sequence in the reference cell population that has not been exposed to the test agent indicates the test agent is a renal protective agent.

The invention also includes a renal protective agent identified according to this screening method, and a pharmaceutical composition which includes the renal protective agent.

Screening for Renal Injury Agents

In one aspect, the invention provides a method of identifying renal injury agents. The renal injury agent can be identified by providing a cell population that includes cells capable of expressing one or more nucleic acid sequences homologous to those listed in Table 1 as RPF 1 - 104. Preferably, the cell population includes cells capable of expressing one or more nucleic acid sequences homologous to RPF 1-104. The sequences need not be identical to sequences including RPF 1-104, as long as the sequence is sufficiently similar that specific hybridization can be detected. Preferably, the cell includes sequences that are identical, or nearly identical to those identifying the RPF nucleic acids shown in Tables 2-4.

Expression of the nucleic acid sequences in the test cell population is then compared to the expression of the nucleic acid sequences in a reference cell population, which is a cell population that has not been exposed to the test agent, or, in some embodiments, a cell population exposed to the test agent. Comparisons can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent, such as an ischemia-inducing compound or process.

An alteration in expression of the nucleic acid sequence in the test cell population compared to the expression of the nucleic acid sequence in the reference cell population that has not been exposed to the test agent indicates the test agent is a renal injury agent.

The invention also includes a renal injury agent identified according to this screening method, and a pharmaceutical composition which includes the renal injury agent.

Methods of treating or preventing Renal Related Disorders

Also included in the invention is a method of treating, i.e., preventing or delaying the onset of renal related disorders in a subject. In various aspects the method includes administering to the subject a compound which modulates the RPF expression or activity. The compound can be, e.g., (i) a RPF polypeptide; (ii) a nucleic acid encoding a RPF polypeptide; (iii) a nucleic acid that increases expression of a nucleic acid that encodes a RPF polypeptide and derivatives, fragments, analogs and homologs thereof. A nucleic acid that increases expression of a nucleic acid that encodes a RPF polypeptide includes, e.g., promoters, enhancers. The nucleic acid can be either endogenous or exogenous. “Modulates” is meant to include an increase or decrease in RPF expression or activity. Preferably, modulation results in alteration of the expression or activity of RPF in a subject to a level similar or identical to a subject not suffering from the renal disorder. In other aspects the method includes administering to the subject a compound which induces a non-renal cell with a renal cell function. In one embodiment the compound modulates RPF expression or activity.

The renal related disorder can be any disorder associated with the kidney. For example, the method may be useful in treating renal hormone insufficiencies, ischemic kidney injury, renal transplantation, drug toxicity, cancer, diabetes, hypertension, glomerulonephritis, childhood lupus nephritis, and polycystic kidney disease. Essentially, any disorder, which is etiologically linked to RPF activity, would be considered susceptible to treatment.

The herein-described RPF modulating compound when used therapeutically are referred to herein as “Therapeutics”. Methods of administration of Therapeutics include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the Therapeutic into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant. Various delivery systems are known and can be used to administer a Therapeutic of the present invention including, e.g.: (i) encapsulation in liposomes, microparticles, microcapsules; (ii) recombinant cells capable of expressing the Therapeutic; (iii) receptor-mediated endocytosis (See, e.g., Wu and Wu, 1987. J Biol Chem 262:4429-4432); (iv) construction of a Therapeutic nucleic acid as part of a retroviral, adenoviral or other vector, and the like. hi one embodiment of the present invention, the Therapeutic may be delivered in a vesicle, in particular a liposome. In a liposome, the protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, all of which are incorporated herein by reference. In yet another embodiment, the Therapeutic can be delivered in a controlled release system including, e.g.: a delivery pump (See, e.g., Saudek, et al., 1989. New Engl JMed 321:574 and a semi-permeable polymeric material (See, e.g., Howard, et al., 1989. JNeurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, FL).

In a specific embodiment of the present invention, where the Therapeutic is a nucleic acid encoding a protein, the Therapeutic nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868), and the like. Alternatively, a nucleic acid Therapeutic can be introduced intracellularly and incorporated within host cell DNA for expression, by homologous recombination or remain episomal.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. However, suitable dosage ranges for intravenous administration of the Therapeutics of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

The duration of intravenous therapy using the Therapeutic of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Cells may also be cultured ex vivo in the presence of therapeutic agents or proteins of the present invention in order to proliferate or to produce a desired effect on or activity in such cells. Treated cells can then be introduced in vivo for therapeutic purposes.

Methods of Identifying Genes Modulated by RPF

The invention also includes a method of identifying nucleic acids modulated by RPF. The method includes measuring the expression of one or more nucleic acids in a test cell population exposed to a compound that modulates RPF activity or expression. Expression of the nucleic acid sequences in the test cell population is then compared to the expression of the nucleic acid sequences in a reference cell population, which is a cell population that has not been exposed to the compound, or, in some embodiments, a cell population exposed to the compound. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of compound can be compared to the expression changes observed in the nucleic acid sequences following administration of a control agent, such as a RPF nucleic acid.

An alteration in expression of the nucleic acid sequence in the test cell population compared to the expression of the nucleic acid sequence in the reference cell population that has not been exposed to the compound indicates expression of the nucleic acid is modulated by RPF.

The test cell can be taken from any tissue capable of being modulated by RPF, e.g., kidney, liver, spleen, or pancreas. In one embodiment the cell is from a non-endocrine tissue. Preferably, the cell is renal tissue.

Preferably, cells in the reference cell population are derived from a tissue type as similar as possible to test cell, e.g., renal tissue. In some embodiments, the control cell is derived from the same subject as the test cell, e.g., from a region proximal to the region of origin of the test cell. In other embodiments, the control cell population is derived from a database of molecular information derived from cells for which the assayed parameter or condition is known.

Expression of the nucleic acids can be measured at the RNA level using any method known in the art. For example, northern hybridization analysis using probes which specifically recognize one or more of these sequences can be used to determine gene expression. Alternatively, expression can be measured using reverse-transcription-based PCR assays. Expression can be also measured at the protein level, i.e., by measuring the levels of polypeptides encoded by the gene products. Such methods are well known in the art and include, e.g., immunoassays based on antibodies to proteins encoded by the genes.

When alterations in gene expression are associated with gene amplification or deletion, sequence comparisons in test and reference populations can be made by comparing relative amounts of the examined DNA sequences in the test and reference cell populations.

The invention also includes RPF modulated nucleic acids identified according to this screening method, and a pharmaceutical composition comprising the RPF modulated nucleic acids so identified.

Assesing Efficacy of Treatment of Renal-Related Disorters in a Subject

The differentially expressed RPF sequences identified herein also allow for the course of treatment of a pathophysiology to be monitored. In this method, a test cell population is provided from a subject undergoing treatment for pathophysiologies associated with RPF expression. If desired, test cell populations can be taken from the subject at various time points before, during, or after treatment. Expression of one or more of the RPF sequences, e.g., RPF 1-104 and, optionally, RPF 3-32 and 103-104, in the cell population is then measured and compared to a reference cell population which includes cells whose pathophysiologic state is known.

Preferably, the reference cells have not been exposed to the treatment.

If the reference cell population contains no cells exposed to the treatment, a similarity in expression between RPF sequences in the test cell population and the reference cell population indicates that the treatment is efficacious. However, a difference in expression between RPF sequences in the test population and this reference cell population indicates the treatment is not efficacious.

By “efficacious” is meant that the treatment leads to a decrease in the pathophysiology in a subject. When treatment is applied prophylactically, “efficacious” means that the treatment retards or prevents a pathophysiology. For example, if the RPF pathophysiology is diabetes, a “efficacious” treatment is one that increases insulin sensitivity in a subject.

Efficaciousness can be determined in association with any known method for treating the particular pathophysiology.

RPF Nucleic Acids

Also provided in the invention are novel nucleic acids comprising a nucleic acid sequence selected from the group consisting of RPF 1-104 or its complement, as well as vectors and cells including these nucleic acids.

Thus, one aspect of the invention pertains to isolated RPF nucleic acid molecules that encode RPF proteins or biologically active portions thereof. Also included are nucleic acid fragments sufficient for use as hybridization probes to identify RPF-encoding nucleic acids (e.g., RPF mRNA) and fragments for use as polymerase chain reaction (PCR) primers for the amplification or mutation of RPF nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt) or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single-or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated RPFnucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of any of RPF 1-104, or a complement of any of these nucleotide sequences, can be isolated using standard molecular biology techniques and the sequence information provided herein. Using all or a portion of these nucleic acid sequences as a hybridization probe, RPFnucleic acid sequences can be isolated using standard hybridization and cloning techniques (e.g., as described in Sambrook et al., eds., molecular cloning: a laboratory manual 2_{nd} Ed., Cold Spring Harbor Laboratory Press, Cold Spring Harbor, NY, 1989; and Ausubel, et al., eds., current protocols in molecular biology, John Wiley & Sons, New York, NY, 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to RPF nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction.

A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having at least about 10 nt and as many as 50 nt, preferably about 15 nt to 30 nt. They may be chemically synthesized and may be used as probes.

In another embodiment, an isolated nucleic acid molecule of the invention comprises a nucleic acid molecule that is a complement of the nucleotide sequence shown in RPF 1- 104. In another embodiment, an isolated nucleic acid molecule of the invention comprises a nucleic acid molecule that is a complement of the nucleotide sequence shown in any of these sequences, or a portion of any of these nucleotide sequences. A nucleic acid molecule that is complementary to the nucleotide sequence shown in RPF 1-b 104 is one that is sufficiently complementary to the nucleotide sequence shown, such that it can hydrogen bond with little or no mismatches to the nucleotide sequences shown, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of RPF 1-104 e.g., a fragment that can be used as a probe or primer or a fragment encoding a biologically active portion of RPF. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 45%, 50%, 70%, 80%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., current Protocols in Molecular Biology , John Wiley & Sons, New York, NY, 1993, and below. An exemplary program in the Gap program (Winsconsin Sequence Analysis Package, Version 8 for UNIX, Genetics Computer Group, University Research Park, Madison, WI) using the default settings, which uses the algorithm of Smith and Waterman (Adv. Appl. Math., 1981, 2: 482-489, which in incorporated herein by reference in its entirety).

A “homologous nucleic acid sequence” or “homologous amino acid sequence,”or variations thereof, refer to sequences characterized by a homology at the nucleotide level or amino acid level as discussed above. Homologous nucleotide sequences encode those sequences coding for isoforms of a RPF polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a RPF polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding a human RPF protein. Homologous nucleic acid sequences include those nucleic acid sequences that encode conservative amino acid substitutions (see below) in a RPF polypeptide, as well as a polypeptide having a RPF activity. A homologous amino acid sequence does not encode the amino acid sequence of a human RPF polypeptide.

The nucleotide sequence determined from the cloning of human RPF genes allows for the generation of probes and primers designed for use in identifying and/or cloning RPF homologues in other cell types, e.g., from other tissues, as well as RPF homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of a nucleic acid comprising a RPF sequence, or an anti-sense strand nucleotide sequence of a nucleic acid comprising a RPF sequence, or of a naturally occurring mutant of these sequences.

Probes based on human RPF nucleotide sequences can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a RPF protein, such as by measuring a level of a RPF-encoding nucleic acid in a sample of cells from a subject e.g., detecting RPF mRNA levels or determining whether a genomic RPF gene has been mutated or deleted.

“A polypeptide having a biologically active portion of RPF” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of RPF” can be prepared by isolating a portion of RPF 1-104, that encodes a polypeptide having a RPF biological activity, expressing the encoded portion of RPF protein (e.g., by recombinant expression in vitro) and assessing the activity of the encoded portion of RPF. For example, a nucleic acid fragment encoding a biologically active portion of a RPF polypeptide can optionally include an ATP-binding domain. In another embodiment, a nucleic acid fragment encoding a biologically active portion of RPF includes one or more regions.

RPF Varias

The invention further encompasses nucleic acid molecules that differ from the disclosed or referenced RPF nucleotide sequences due to degeneracy of the genetic code. These nucleic acids thus encode the same RPF protein as that encoded by nucleotide sequence comprising a RPF nucleic acid as shown in, e.g., RPF 1-104

In addition to the mouse RPF nucleotide sequence shown in RPF 1-104, it will be appreciated by those skilled in the art that DNA sequence polymorphisms that lead to changes in the amino acid sequences of a RPF polypeptide may exist within a population (e.g., the human population). Such genetic polymorphism in the RPF gene may exist among individuals within a population due to natural allelic variation. As used herein, the terms “gene” and “recombinant gene” refer to nucleic acid molecules comprising an open reading frame encoding a RPF protein, preferably a mammalian RPF protein. Such natural allelic variations can typically result in 1-5% variance in the nucleotide sequence of the RPF gene. Any and all such nucleotide variations and resulting amino acid polymorphisms in RPF that are the result of natural allelic variation and that do not alter the functional activity of RPF are intended to be within the scope of the invention.

Moreover, nucleic acid molecules encoding RPF proteins from other species, and thus that have a nucleotide sequence that differs from the human sequence of RPF 1-104, are intended to be within the scope of the invention. Nucleic acid molecules corresponding to natural allelic variants and homologues of the RPF DNAs of the invention can be isolated based on their homology to the human RPF nucleic acids disclosed herein using the human cDNAs, or a portion thereof, as a hybridization probe according to standard hybridization techniques under stringent hybridization conditions. For example, a soluble human RPF DNA can be isolated based on its homology to human membrane-bound RPF. Likewise, a membrane-bound human RPF DNA can be isolated based on its homology to soluble human RPF.

Accordingly, in another embodiment, an isolated nucleic acid molecule of the invention is at least 6 nucleotides in length and hybridizes under stringent conditions to the nucleic acid molecule comprising the nucleotide sequence of RPF 1-104. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding RPF proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides.

Stringent conditions may also be achieved with the addition of destabilizing agents, such as formamide.

Stringent conditions are known to those skilled in the art and can be found in current protocols in molecular biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6X SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of RPF 1-104 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of RPF 1-104 or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, current protocols in molecular biology, John Wiley & Sons, NY, and Kriegler, 1990, gene transfer and expression, a laboratory manual, Stockton Press, NY.

In a third embodiment, a nucleic acid that is hybridizable to the nucleic acid molecule comprising the nucleotide sequence of RPF 1-104or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10 % (wt/vol) dextran sulfate at 40° C., followed by one or more washes in 2×SSC, 25 mM Tris-HCl (pH 7.4), 5 mM EDTA, and 0. 1% SDS at 50° C. Other conditions of low stringency that may be used are well known in the art (e.g., as employed for cross-species hybridizations). See, e.g., Ausubel et al. (eds.), 1993, current protocols in molecular bilogy,John Wiley & Sons, NY, and Kriegler, 1990, gene transfer and expression, a laboratory manual, Stockton Press, NY; Shilo et al., 1981, Proc Natl Acad Sci USA 78: 6789-6792.

Conservative Mutations

In addition to naturally-occurring allelic variants of the RPF sequence that may exist in the population, the skilled artisan will further appreciate that changes can be introduced into an RPFnucleic acid or directly into an RPF polypeptide sequence without altering the functional ability of the RPF protein. In some embodiments, the nucleotide sequence of RPF 1-104will be altered, thereby leading to changes in the amino acid sequence of the encoded RPF protein. For example, nucleotide substitutions that result in amino acid substitutions at various “non-essential” amino acid residues can be made in the sequence of RPF 1-104. A “non-essential” amino acid residue is a residue that can be altered from the wild-type sequence of RPF without altering the biological activity, whereas an “essential” amino acid residue is required for biological activity. For example, amino acid residues that are conserved among the RPFproteins of the present invention, are predicted to be particularly unamenable to alteration.

In addition, amino acid residues that are conserved among family members of the RPF proteins of the present invention, are also predicted to be particularly unamenable to alteration. As such, these conserved domains are not likely to be amenable to mutation. Other amino acid residues, however, (e.g., those that are not conserved or only semi-conserved among members of the RPF proteins) may not be essential for activity and thus are likely to be amenable to alteration.

Another aspect of the invention pertains to nucleic acid molecules encoding RPF proteins that contain changes in amino acid residues that are not essential for activity. Such RPF proteins differ in amino acid sequence from the amino acid sequences of polypeptides encoded by nucleic acids containing RPF 1-104, yet retain biological activity. In one embodiment, the isolated nucleic acid molecule comprises a nucleotide sequence encoding a protein, wherein the protein comprises an amino acid sequence at least about 45% homologous, more preferably 60%, and still more preferably at least about 70%, 80%, 90%, 95%, 98%, and most preferably at least about 99% homologous to the amino acid sequence of the amino acid sequences of polypeptides encoded by nucleic acids comprising RPF 1-104.

An isolated nucleic acid molecule encoding a RPF protein homologous to can be created by introducing one or more nucleotide substitutions, additions or deletions into the nucleotide sequence of a nucleic acid comprising RPF 1- 104, such that one or more amino acid substitutions, additions or deletions are introduced into the encoded protein.

Mutations can be introduced into a nucleic acid comprising RPF 1-104 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in RPF is replaced with another amino acid residue from the same side chain family. Alternatively, in another embodiment, mutations can be introduced randomly along all or part of a RPF coding sequence, such as by saturation mutagenesis, and the resultant mutants can be screened for RPF biological activity to identify mutants that retain activity. Following mutagenesis of the nucleic acid, the encoded protein can be expressed by any recombinant technology known in the art and the activity of the protein can be determined.

In one embodiment, a mutant RPF protein can be assayed for (1) the ability to form protein: protein interactions with other RPF proteins, other cell-surface proteins, or biologically active portions thereof, (2) complex formation between a mutant RPF protein and a RPF ligand; (3) the ability of a mutant RPF protein to bind to an intracellular target protein or biologically active portion thereof; (e.g., avidin proteins); (4) the ability to bind ATP; or (5) the ability to specifically bind a RPF protein antibody.

Antisense

Another aspect of the invention pertains to isolated antisense nucleic acid molecules that are hybridizable to or complementary to the nucleic acid molecule comprising the nucleotide sequence of a RPF sequence or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an MRNA sequence. In specific aspects, antisense nucleic acid molecules are provided that comprise a sequence complementary to at least about 10, 25, 50, 100, 250 or 500 nucleotides or an entire RPF coding strand, or to only a portion thereof. Nucleic acid molecules encoding fragments, homologs, derivatives and analogs of a RPF protein, or antisense nucleic acids complementary to a nucleic acid comprising a RPF nucleic acid sequence are additionally provided.

In one embodiment, an antisense nucleic acid molecule is antisense to a “coding region” of the coding strand of a nucleotide sequence encoding RPF. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding RPF. The term “noncoding region” refers to 5′and 3′sequences which flank the coding region that are not translated into amino acids (i.e., also referred to as 5′ and 3′ untranslated regions).

Given the coding strand sequences encoding RPF disclosed herein, antisense nucleic acids of the invention can be designed according to the rules of Watson and Crick or Hoogsteen base pairing. The antisense nucleic acid molecule can be complementary to the entire coding region of RPF mRNA, but more preferably is an oligonucleotide that is antisense to only a portion of the coding or noncoding region of RPF MRNA. For example, the antisense oligonucleotide can be complementary to the region surrounding the translation start site of RPF mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxyrnethylaminomethyluracil, dihydrouracil, beta-D-galactosylqueosine, inosine, N6-isopentenyladenine, 1-methylguanine, 1-methylinosine, 2,2-dimethylguanine, 10 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, -beta-D-mannosylqueosine, 5′-methoxycarboxymethyluracil, 5-methoxyuracil, -2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a RPF protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA -DNA analogue (Inoue et al. (1987) FEBS Lett 215: 327-330).

Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave RPF mRNA transcripts to thereby inhibit translation of RPF mRNA. A ribozyme having specificity for a RPF-encoding nucleic acid can be designed based upon the nucleotide sequence of a RPF DNA disclosed herein. For example, a derivative of a Tetrahymena L-19 IVS RNA can be constructed in which the nucleotide sequence of the active site is complementary to the nucleotide sequence to be cleaved in a RPF-encoding mRNA. See, e.g., Cech et al. U.S. Pat. No. 4,987,071; and Cech et al. U.S. Pat. No. 5,116,742. Alternatively, RPF mRNA can be used to select a catalytic RNA having a specific ribonuclease activity from a pool of RNA molecules. See, e.g., Bartel et al., (1993) Science 261:1411-1418.

Alternatively, RPF gene expression can be inhibited by targeting nucleotide sequences complementary to the regulatory region of a RPF nucleic acid (e.g., the RPF promoter and/or enhancers) to form triple helical structures that prevent transcription of the RPF gene in target cells. See generally, Helene. (1991) Anticancer Drug Des. 6: 569-84; Helene. et al. (1992) Ann. N. Y Acad. Sci. 660:27-36; and Maher (1992) Bioassays 14: 807-15.

In various embodiments, the nucleic acids of RPF can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of RPF can be used in therapeutic and diagnostic applications. For example, PNAs can be used as antisense or antigene agents for sequence-specific modulation of gene expression by, e.g., inducing transcription or translation arrest or inhibiting replication. PNAs of RPF can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of RPF can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of RPF can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) BioorgMed Chem Lett5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556;

Lemaitre et al., 1987, Proc. Natl. Acad. Sci. 84:648-652; PCT Publication No. W088/09810) or the blood-brain barrier (see, e.g., PCT Publication No. W089/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

RPF Polypeptides

One aspect of the invention pertains to isolated RPF proteins, and biologically active portions thereof, or derivatives, fragments, analogs or homologs thereof. Also provided are polypeptide fragments suitable for use as immunogens to raise anti-RPF antibodies. In one embodiment, native RPF proteins can be isolated from cells or tissue sources by an appropriate purification scheme using standard protein purification techniques. In another embodiment, RPF proteins are produced by recombinant DNA techniques. Alternative to recombinant expression, a RPF protein or polypeptide can be synthesized chemically using standard peptide synthesis techniques.

An “isolated” or “purified” protein or biologically active portion thereof is substantially free of cellular material or other contaminating proteins from the cell or tissue source from which the RPF protein is derived, or substantially free from chemical precursors or other chemicals when chemically synthesized. The language “substantially free of cellular material” includes preparations of RPF protein in which the protein is separated from cellular components of the cells from which it is isolated or recombinantly produced. In one embodiment, the language “substantially free of cellular material” includes preparations of RPF protein having less than about 30% (by dry weight) of non-RPF protein (also referred to herein as a “contaminating protein”), more preferably less than about 20% of non-RPF protein, still more preferably less than about 10% of non-RPF protein, and most preferably less than about 5% non-RPF protein. When the RPF protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of RPF protein in which the protein is separated from chemical precursors or other chemicals that are involved in the synthesis of the protein. In one embodiment, the language “substantially free of chemical precursors or other chemicals” includes preparations of RPF protein having less than about 30% (by dry weight) of chemical precursors or non-RPF chemicals, more preferably less than about 20% chemical precursors or non-RPF chemicals, still more preferably less than about 10% chemical precursors or non-RPF chemicals, and most preferably less than about 5% chemical precursors or non-RPFchemicals.

Biologically active portions of a RPF protein include peptides comprising amino acid sequences sufficiently homologous to or derived from the amino acid sequence of the RPF protein, e.g., the amino acid sequence encoded by a nucleic acid comprising RPF 1-20 that include fewer amino acids than the full length RPF proteins, and exhibit at least one activity of a RPF protein. Typically, biologically active portions comprise a domain or motif with at least one activity of the RPF protein. A biologically active portion of a RPF protein can be a polypeptide which is, for example, 10, 25, 50, 100 or more amino acids in length.

A biologically active portion of a RPF protein of the present invention may contain at least one of the above-identified domains conserved between the RPF proteins. An alternative biologically active portion of a RPF protein may contain at least two of the above-identified domains. Another biologically active portion of a RPF protein may contain at least three of the above-identified domains. Yet another biologically active portion of a RPF protein of the present invention may contain at least four of the above-identified domains.

Moreover, other biologically active portions, in which other regions of the protein are deleted, can be prepared by recombinant techniques and evaluated for one or more of the functional activities of a native RPF protein.

In some embodiments, the RPF protein is substantially homologous to one of these RPF proteins and retains its the finctional activity, yet differs in amino acid sequence due to natural allelic variation or mutagenesis, as described in detail below.

In specific embodiments, the invention includes an isolated polypeptide comprising an amino acid sequence that is 80% or more identical to the sequence of a polypeptide whose expression is modulated in a mammal to which renal toxic agent is administered.

Determining Homology between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of a DNA sequence comprising RPF: :1-7, 10-13, 19-34, 45-53, 58-85, 111-113, 120, 130, 132-134 and 138.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides RPF chimeric or fusion proteins. As used herein, an RPF “chimeric protein” or “fusion protein” comprises an RPF polypeptide operatively linked to a non-RPF polypeptide. A “RPF polypeptide” refers to a polypeptide having an amino acid sequence corresponding to RPF, whereas a “non-RPF polypeptide” refers to a polypeptide having an amino acid sequence corresponding to a protein that is not substantially homologous to the RPF protein, e.g., a protein that is different from the RPF protein and that is derived from the same or a different organism. Within an RPF fusion protein the RPF polypeptide can correspond to all or a portion of an RPF protein. In one embodiment, an RPF fusion protein comprises at least one biologically active portion of an RPF protein. In another embodiment, an RPF fusion protein comprises at least two biologically active portions of an RPF protein. In yet another embodiment, an RPF fusion protein comprises at least three biologically active portions of an RPF protein. Within the fusion protein, the term “operatively linked” is intended to indicate that the RPF polypeptide and the non-RPF polypeptide are fused in-frame to each other. The non-RPF polypeptide can be fused to the N-terminus or C-terminus of the RPF polypeptide.

For example, in one embodiment an RPF fusion protein comprises an RPF domain operably linked to the extracellular domain of a second protein. Such fusion proteins can be further utilized in screening assays for compounds which modulate RPF activity (such assays are described in detail below).

In yet another embodiment, the fusion protein is a GST-RPF fusion protein in which the RPF sequences are fused to the C-terminus of the GST (i.e., glutathione S-transferase) sequences.

Such fusion proteins can facilitate the purification of recombinant RPF.

In another embodiment, the fusion protein is an RPF protein containing a heterologous signal sequence at its N-terminus. For example, a native RPF signal sequence can be removed and replaced with a signal sequence from another protein. In certain host cells (e.g., mammalian host cells), expression and/or secretion of RPF can be increased through use of a heterologous signal sequence.

In yet another embodiment, the fusion protein is an RPF-immunoglobulin fusion protein in which the RPF sequences comprising one or more domains are fused to sequences derived from a member of the immunoglobulin protein family. The RPF-immunoglobulin fusion proteins of the invention can be incorporated into pharmaceutical compositions and administered to a subject to inhibit an interaction between a RPF ligand and a RPF protein on the surface of a cell, to thereby suppress RPF-mediated signal transduction in vivo. The RPF-immunoglobulin fusion proteins can be used to affect the bioavailability of an RPF cognate ligand. Ihibition of the RPF ligand/RPF interaction may be useful therapeutically for both the treatments of proliferative and differentiative disorders, as well as modulating (e.g. promoting or inhibiting) cell survival. Moreover, the RPF-immunoglobulin fusion proteins of the invention can be used as immunogens to produce anti-RPF antibodies in a subject, to purify RPF ligands, and in screening assays to identify molecules that inhibit the interaction of RPF with a RPF ligand.

An RPF chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) current protocols in molecular biology, John Wiley & Sons, 1992). Moreover, many expression vectors are commercially available that already encode a fusion moiety (e.g., a GST polypeptide). An RPF-encoding nucleic acid can be cloned into such an expression vector such that the fusion moiety is linked in-frame to the RPF protein.

RPF Agonists and Antagonists

The present invention also pertains to variants of the RPF proteins that function as either RPF agonists (mimetics) or as RPF antagonists. Variants of the RPF protein can be generated by mutagenesis, e.g., discrete point mutation or truncation of the RPF protein. An agonist of the RPF protein can retain substantially the same, or a subset of, the biological activities of the naturally occurring form of the RPF protein. An antagonist of the RPF protein can inhibit one or more of the activities of the naturally occurring form of the RPF protein by, for example, competitively binding to a downstream or upstream member of a cellular signaling cascade which includes the RPF protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the RPF proteins.

Variants of the RPF protein that finction as either RPF agonists (mimetics) or as RPF antagonists can be identified by screening combinatorial libraries of mutants, e.g., truncation mutants, of the RPF protein for RPF protein agonist or antagonist activity. In one embodiment, a variegated library of RPF variants is generated by combinatorial mutagenesis at the nucleic acid level and is encoded by a variegated gene library. A variegated library of RPF variants can be produced by, for example, enzymatically ligating a mixture of synthetic oligonucleotides into gene sequences such that a degenerate set of potential RPF sequences is expressible as individual polypeptides, or alternatively, as a set of larger fusion proteins (e.g., for phage display) containing the set of RPF sequences therein. There are a variety of methods which can be used to produce libraries of potential RPF variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential RPF sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the RPF protein coding sequence can be used to generate a variegated population of RPF fragments for screening and subsequent selection of variants of an RPF protein. In one embodiment, a library of coding sequence fragments can be generated by treating a double stranded PCR fragment of a RPF coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with SI nuclease, and ligating the resulting fragment library into an expression vector. By this method, an expression library can be derived which encodes N-terminal and internal fragments of various sizes of the RPF protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of RPF proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify RPF variants (Arkin and Yourvan (1992) PNAS 89:7811-7815; Delgrave et al. (1993) Protein Engineering 6:327-331).

Anti-NTI-RPF ANTIBODIES

An isolated RPF protein, or a portion or fragment thereof, can be used as an immunogen to generate antibodies that bind RPF using standard techniques for polyclonal and monoclonal antibody preparation. The full-length RPF protein can be used or, alternatively, the invention provides antigenic peptide fragments of RPF for use as immunogens. The antigenic peptide of RPF comprises at least 8 amino acid residues of the amino acid sequence encoded by a nucleic acid comprising the nucleic acid sequence shown in RPF 1-104 and encompasses an epitope of RPF such that an antibody raised against the peptide forms a specific immune complex with RPF. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of RPF that are located on the surface of the protein, e.g., hydrophilic regions. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 5 78: 3824-3828; Kyte and Doolittle 1982, J. Mol. Biol. 157: 105-142, each incorporated herein by reference in their entirety.

RPF polypeptides or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen. Such antibodies include, but are not limited to, polyclonal, monoclonal, chimeric, single chain, Fab and F(ab,)2 fragments, and an Fab expression library. Various procedures known within the art may be used for the production of polyclonal or monoclonal antibodies to an RPF protein sequence, or derivatives, fragments, analogs or homologs thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed RPF protein or a chemically synthesized RPF polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against RPF can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of RPF. A monoclonal antibody composition thus typically displays a single binding affinity for a particular RPF protein with which it immunoreacts. For preparation of monoclonal antibodies directed towards a particular RPF protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, 10 et al., 1983 Immunol Today 4: 72) and the EBV hybridoma technique to produce human monoclonal antibodies (see Cole, et al., 1985 In: monoclonal antibodies and cancer therapy, Alan R.Liss, Inc.,pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: monoclonal antibodies and cancer therapy Alan R. Liss, Inc., pp. 77-96).

According to the invention, techniques can be adapted for the production of single-chain antibodies specific to a RPF protein (see e.g., U.S. Pat. No. 4,946,778). In addition, methods can be adapted for the construction of Fab expression libraries (see e.g., Huse, et aL, 1989 Science 246: 1275-1281) to allow rapid and effective identification of monoclonal F_{ab} fragments with the desired specificity for a RPF protein or derivatives, fragments, analogs or homologs thereof.

Non-human antibodies can be “humanized” by techniques well known in the art. See e.g., U.S. Pat. No. 5,225,539. Antibody fragments that contain the idiotypes to a RPF protein may be produced by techniques known in the art including, but not limited to: (i) an F(ab)2 fragment produced by pepsin digestion of an antibody molecule; (ii) an Fab fragment generated by reducing the disulfide bridges of an F(_{ab′)2} fragment; (iii) an F_{ab} fragment generated by the treatment of the antibody molecule with papain and a reducing agent and (iv) Fᵥ, fragments.

Additionally, recombinant anti-RPF antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst. 80:1553-1559); Morrison(1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) JImmunol 141:4053-4060.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a RPF protein is facilitated by generation of hybridomas that bind to the fragment of a RPF protein possessing such a domain. Antibodies that are specific for one or more domains within a RPF protein, e.g., domains spanning the above-identified conserved regions of RPF family proteins, or derivatives, fragments, analogs or homologs thereof, are also provided herein.

Anti-RPF antibodies may be used in methods known within the art relating to the localization and/or quantitation of a RPF protein (e.g., for use in measuring levels of the RPF protein within appropriate physiological samples, for use in diagnostic methods, for use in imaging the protein, and the like). In a given embodiment, antibodies for RPF proteins, or derivatives, fragments, analogs or homologs thereof, that contain the antibody derived binding domain, are utilized as pharmacologically-active compounds [hereinafter “Therapeutics”].

An anti-RPF antibody (e.g., monoclonal antibody) can be used to isolate RPF by standard techniques, such as affinity chromatography or immunoprecipitation. An anti-RPF antibody can facilitate the purification of natural RPF from cells and of recombinantly produced RPF expressed in host cells. Moreover, an anti-RPF antibody can be used to detect RPF protein (e.g., in a cellular lysate or cell supernatant) in order to evaluate the abundance and pattern of expression of the RPF protein. Anti-RPF antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/ibiotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S, or 3 H. luciferin, and aequorin, and examples of suitable radioactive material include I, I, S or ³H.

RPF Recombinant Expression Vectors and Host Cells

Another aspect of the invention pertains to vectors, preferably expression vectors, containing a nucleic acid encoding RPF protein, or derivatives, fragments, analogs or homologs thereof. As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; gene expression technoloy: methods in ezymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., RPF proteins, mutant forms of RPF, fusion proteins, etc.).

The recombinant expression vectors of the invention can be designed for expression of RPF in prokaryotic or eukaryotic cells. For example, RPF can be expressed in bacterial cells such as E. coli, insect cells (using baculovirus expression vectors) yeast cells or mammalian cells. Suitable host cells are discussed further in Goeddel, gene expression technology:

METHODS INENZYMoLoGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc;

Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11 Id (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPREssION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:211:1-7, 10-13, 19-34, 45-53, 58-85, 111-113, 120, 130, 132-134 and 13518). Such alteration of nucleic acid sequences of the invention can be carried out by standard DNA synthesis techniques.

In another embodiment, the RPF expression vector is a yeast expression vector. Examples of vectors for expression in yeast S. cerevisiae include pYepSecl (Baldari, et al., (1987) EMBO J6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp, San Diego, Calif.).

Alternatively, RPF can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8(Seed (1987) Nature 329:840) and pMT2PC (Kaufinan et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), kidney-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to RPF mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews--Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, RPF protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding RPF or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an RPF protein. Accordingly, the invention further provides methods for producing RPF protein using the host cells of the invention. In one embodiment, the method comprises culturing the host cell of invention (into which a recombinant expression vector encoding RPF has been introduced) in a suitable medium such that RPF protein is 25 produced. In another embodiment, the method further comprises isolating RPF from the medium or the host cell.

### PHARMACEUTICAL COMPOSITIONS

The RPF nucleic acid molecules, RPF proteins, and anti-RPF antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifingal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a RPF protein or anti-RPF antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### KITS AND NUCLEIC ACID COLLECTIONS FOR IDENTIFYING RPF NUCLEIC ACIDS

In another aspect, the invention provides a kit useful for examining renal toxicity of agents. The kit can include nucleic acids that detect two or more RPF sequences. In preferred embodiments, the kit includes reagents which detect 3, 4, 5, 6, 8, 10, 12, 15, 20, 25, 50, 100 or all of the RPF nucleic acid sequences.

The invention also includes an isolated plurality of sequences which can identify one or more RPF responsive nucleic acid sequences.

The kit or plurality may include, e.g., sequence homologous to RPF nucleic acid sequences, or sequences which can specifically identify one or more RPF nucleic acid sequences.

### NUCLEOTIDE POLYMORPHISMS ASSOCIATED WITH RPF GENES

The invention also includes nucleic acid sequences that include one or more polymorphic RPF sequences. Also included are methods of identifying a base occupying a polymorphic in an RPF sequence, as well as methods of identifying an individualized therapeutic agent for treating renal injury agent associated pathologies, e.g., valvular kidney disease, pulminary hypertention, coronary vasospasm, or valvular and peripheral fibrosis based on RPF sequence polymorphisms.

The nucleotide polymorphism can be a single nucleotide polymorphism (SNP). A SNP occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. In some embodiments, the polymorphic sequence includes the full length of any one of the RPF genes in Tables 2-4. In other embodiments, the polymorphic sequence includes a polynucleotide that is between 10 and 100 nucleotides, 10 and 75 nucleotides, 10 and 50 nucleotides, or 10 and 25 nucleotides in length.

The invention also provides a method of identifying a base occupying a polymorphic site in a nucleic acid. The method includes determining the nucleotide sequence of a nucleic acid that is obtained from a subject. The nucleotide sequence is compared to a reference sequence. Difference in the nucleotide sequence in the test sequence relative to the reference sequence indicates a polymorphic site in the nucleic acid.

Polymorphisms are detected in a target nucleic acid from an individual, e.g., a mammal, human or rodent (such as mouse or rat) being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed.

The detection of polymorphisms in specific DNA sequences, can be accomplished by a variety of methods including, e.g., restriction-fragrnent-length-polymorphism detection based on allele-specific restriction-endonuclease cleavage (Kan and Dozy Lancet ii:910-912 (1978)), hybridization with allele-specific oligonucleotide probes (Wallace et al. Nucl. Acids Res. 6:3543-3557 (1978)), including immobilized oligonucleotides (Saiki et al. Proc. Natl. Acad.

SCI. USA, 86:6230-6234 (1969)) or oligonucleotide arrays (Maskos and Southern Nucl. Acids Res 21:2269-2270 (1993)), allele-specific PCR (Newton et al. Nucl Acids Res 17:2503-2516 (1989)), mismatch-repair detection (MRD) (Faham and Cox Genome Res 5:474-482 (1995)), binding of MutS protein (Wagner et al. Nucl Acids Res 23:3944-3948 (1995), denaturing-gradient gel electrophoresis (DGGE) (Fisher and Lerman et al. Proc. Nati. Acad. Sci. U.S.A. 80:1579-l 583 (1983)), single-strand-conformation-polymorphism detection (Orita et al.

Genomics 5:874-879 (1983)), RNAase cleavage at mismatched base-pairs (Myers et al. Science 230:1242 (1985)), chemical (Cotton et al. Proc. Natl. w Sci. U.S.A, 8Z4397-4401 (1988)) or enzymatic (Youil et al. Proc. Natl. Acad. Sci. U.S.A. 92:87-91 (1995)) cleavage of heteroduplex DNA, methods based on allele specific primer extension (Syvanen et al. Genomics 8:684-692 (1990)), genetic bit analysis (GBA) (Nikiforov et al. &&I Acids 22:4167-4175 (1994)), the oligonucleotide-ligation assay (OLA) (Landegren et al. Science-241:1077 (1988)), the allele-specific ligation chain reaction (LCR) (Barrany Proc. Natl. Acad. Sci. U.S.A. 88:189-193 (1991)), gap-LCR (Abravaya et al. Nucl Acids Res 23:675-682 (1995)), radioactive and/or fluorescent DNA sequencing using standard procedures well known in the art, and peptide nucleic acid (PNA) assays (Orum et al., Nucl. Acids Res, 21:5332-5356 (1993); Thiede et al., Nucl. Acids Res. 24:983-984 (1996)).

For the purposes of identifying single nucleotide polymorphisms, “Specific hybridization” or “selective hybridization” refers to the binding, or duplexing, of a nucleic acid molecule only to a second particular nucleotide sequence to which the nucleic acid is complementary, under suitably stringent conditions when that sequence is present in a complex mixture (e.g., total cellular DNA or RNA). “Stringent conditions” are conditions under which a probe will hybridize to its target subsequence, but to no other sequences. Stringent conditions are sequence-dependent and are different in different circumstances. Longer sequences hybridize specifically at higher temperatures than shorter ones. Generally, stringent conditions are selected such that the temperature is about 5° C. lower than the thermal melting point (Tm) for the specific sequence to which hybridization is intended to occur at a defined ionic strength and pH. The Tm is the temperature (under defmed ionic strength, pH, and nucleic acid concentration) at which 50% of the target sequence hybridizes to the complementary probe at equilibrium. Typically, stringent conditions include a salt concentration of at least about 0.01 to about 1.0 M Na ion concentration (or other salts), at pH 7.0 to 8.3. The temperature is at least about 30° C for short probes (e.g., 10 to 50 nucleotides) . Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations.

“Complementary” or “target” nucleic acid sequences refer to those nucleic acid sequences which selectively hybridize to a nucleic acid probe. Proper annealing conditions depend, for example, upon a probe's length, base composition, and the number of mismatches and their position on the probe, and must often be determined empirically. For discussions of nucleic acid probe design and annealing conditions, see, for example, Sambrook et al., or Current Protocols in Molecular Biology, F. Ausubel et al., ed., Greene Publishing and Wiley-Interscience, New York 25 (1987).

Many of the methods described above require amplification of DNA from target samples.

This can be accomplished by e.g., PCR. See generally, PCR Technology: Principles and Applications for DNA Amplification (ed. H. A. Erlich, Freeman Press, N.Y., N.Y., 1992); PCR Protocols: A Guide to Methods and Applications (eds. Inis, et al., Academic Press, San Diego, Calif., 1990); Mattila et al., Nucleic Acids Res. 19, 4967 (1991); Eckert et al, PCR Methods and Applications 1, 17 (1991); PCR (eds. McPherson et al., IRL Press, Oxford); and U.S. Pat. No. 4,683,202 (each of which is incorporated by reference for all purposes).

Other suitable amplification methods include the ligase chain reaction (LCR), (See Wu and Wallace, Genomics 4, 560 (1989), Landegren et al.., Science 241, 1077 (1988)), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The invention also provides a method of selecting an individualized therapeutic agent for treating a renal injury agent associated pathology, e.g., valvular kidney disease, pulmonary hypertension, in a subject using RPF polymorphisms. The therapeutic agent can be identified by providing a nucleic acid sample from the subject, determining the nucleotide sequence of at least a portion of one or more of the RPF 1-104 and comparing the RPF nucleotide sequence in the subject to the corresponding RPF nucleic acid sequence in a reference nucleic acid sample. The reference nucleic acid sample is obtained from a reference individual (who is preferably as similar to the test subject as possible), whose responsiveness to the agent for treating the renal injury agent associated pathology is known. The presence of the same sequence in the test and reference nucleic acid sample indicates the subject will demonstrate the same responsiveness to the agent as the reference individual, while the presence of a different sequence indicates the subject will have a different response to the therapeutic agent.

Similarly, the RPF-associated sequence polymorphisms can be used to predict the outcome of treatment for a renal injury agent associated pathology, e.g., valvular kidney disease, pulmonary hypertension, in a subject. A region of a RPF nucleic acid sequence from the subject is compared to the corresponding RPF sequence in a reference individual whose outcome in response to the treatment for the renal injury agent associated pathology is known. A similarity in the RPF sequence in the test subject as compared to the sequence in the reference individual suggests the outcome in the subject will be the same as that of the reference individual. An altered RPF sequence in the test and reference individual indicates the outcome of treatment will differ in the subject and reference individuals.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a renal protective agent, the method comprising;
(a) providing a test cell population comprising a cell capable of expressing one or more nucleic acid sequences selected from the group consisting of RPF 1- 104;
(b) contacting the test cell population with a test agent;
(c) measuring expression of one or more of the nucleic acid sequences in the test cell population;
(d) comparing the expression of the nucleic acid sequences in the test cell population to the expression of the nucleic acid sequences in a reference cell population comprising at least one cell whose renal status is known; and
(e) identifying a difference in expression levels of the RPF sequence, if present, in the test cell population and reference cell population, thereby identifying a renal protective agent.

2. The method of claim 1, wherein the method comprises comparing the expression of one or more genes selected from the group consisting of RPF 3-32 and 103- 104.

3. The method of claim 1, wherein the method comprises comparing the expression of one or more genes selected from the group consisting of RPF 3-23.

4. The method of claim 3, wherein the expression of the nucleic acid sequences in the test cell population is increased as compared to the reference cell population.

5. The method of claim 2, wherein the expression of the nucleic acid sequences in the test cell population is decreased as compared to the reference cell population.

6. The method of claim 1, wherein the test cell population is provided in vitro.

7. The method of claim 1, wherein the test cell population is provided ex vivo from a mammalian subject.

8. The method of claim 1, wherein the test cell population is provided in vivo in a mammalian subject.

9. The method of claim 1, wherein the test cell population is derived from a human or rodent subject.

10. The method of claim 1, wherein the test cell population includes a kidney cell.

11. The method of claim 10, wherein said test cell population is selected from the group consisting of mesangial cells, endothelial cells, glomerular cells, renal epithelial cells, embryonic kidney cells, and renal tubular cells.

12. The method of claim 1, wherein the method comprises comparing the expression of five or more of the nucleic acid sequences.

13. The method of claim 1, wherein the method comprises comparing the expression of 20 or more of the nucleic acid sequences.

14. The method of claim 1, wherein the method comprises comparing the expression of 25 or more of the nucleic acid sequences.

15. A method of screening a test agent for renal toxicity, the method comprising;
(a) providing a test cell population comprising a cell capable of expressing one or more nucleic acid sequences selected from the group consisting of RPF 1 - 104;
(b) contacting the test cell population with a test agent;
(c) measuring expression of one or more of the nucleic acid sequences in the test cell population;
(d) comparing the expression of the nucleic acid sequences in the test cell population to the expression of the nucleic acid sequences in a reference cell population comprising at least one cell whose renal toxic agent expression status is known; and
(e) identifying a difference in expression levels of the RPF sequence, if present, in the test cell population and reference cell population, thereby screening said test agent for renal toxicity.

16. A method of diagnosing or determining the susceptibility to renal injury in a subject, the method comprising:
(a) providing from the subject a test cell population comprising a cell capable of expressing one or more nucleic acid sequences selected from the group consisting of RPF 1-104;
(b) contacting the test cell population with a test agent, said agent being capable of altering expression of one or more of the nucleic acid sequences in the test cell population which are altered during renal injury;
(c) measuring the expression of one or more of the nucleic acid sequences in the test cell population;
(d) comparing the expression of the nucleic acid sequences in the test cell population to the expression of the nucleic acid sequences in a reference cell population comprising at least one cell whose renal injury agent expression status is known; and
(e) identifying a difference in expression levels of the RPF sequence, if present, in the test cell population and reference cell population, thereby diagnosing or determining the susceptibility to renal injury in the subject.

17. The method of claim 16, wherein said renal injury is selected from the group consisting of ischemic kidney injury, renal transplantation, drug toxicity, cancer, diabetes, hypertension, childhood lupus nephritis, and polycystic kidney disease.

18. A method of assessing the renal protective effect of a test agent in a subject, the method comprising:
(a) providing from the subject a test cell population comprising a cell capable of expressing one or more nucleic acid sequences selected from the group consisting of RPF 1-104;
(b) contacting the test cell population with a test agent;
(c) measuring expression of one or more of the nucleic acid sequences in the test cell population; and
(d) comparing the expression of the nucleic acid sequences in the test cell population to the expression of the nucleic acid sequences in a reference cell population comprising at least one cell whose renal injury agent expression status is known;
(e) identifying a difference in expression levels of the nucleic acid sequences, if present, in the test cell population and the reference cell population, thereby assessing the renal protective effect of the test agent in the subject.

19. The method of claim 18, wherein the method comprises comparing the expression of one or more genes selected from the group consisting of RPF 3-32 and 103- 104.

20. The method of claim 18, wherein the method comprises comparing the expression of one or more genes selected from the group consisting of RPF 3-23 and 103- 104.

21. The method of claim 20, wherein the expression of the nucleic acid sequences in the test cell population is increased as compared to the reference cell population.

22. The method of claim 19, wherein the expression of the nucleic acid sequences in the test cell population is decreased as compared to the reference cell population.

23. The method of claim 18, wherein said subject is a human or rodent.

24. The method of claim 18, wherein the test cell population is provided ex vivo from said subject.

25. The method of claim 18, wherein the test cell population is provided in vivo from said subject.

26. A method of assessing the renal toxicity of a test agent in a subject, the method comprising:
(a) providing from the subject a test cell population comprising a cell capable of expressing one or more nucleic acid sequences selected from the group consisting of RPF 1-104;
(b) contacting the test cell population with a test agent;
(c) measuring expression of one or more of the nucleic acid sequences in the test cell population; and
(d) comparing the expression of the nucleic acid sequences in the test cell population to the expression of the nucleic acid sequences in a reference cell population comprising at least one cell whose renal toxic agent expression status to a renal toxic agent is known;
(e) identifying a difference in expression levels of the nucleic acid sequences, if present, in the test cell population and the reference cell population, thereby assessing the renal toxicity of the test agent in the subject.

27. A method of a treating a renal related disorder in a subject, said method comprising administering to a subject in need thereof a therapeutically effective amount of a compound which modulates RPF expression or activity in said subject, thereby treating said renal disorder in said subject.

28. The method of claim 27, wherein said renal disorder is selected from the group consisting of ischemic kidney injury, renal transplantation, drug toxicity, cancer, diabetes, hypertension, childhood lupus nephritis, and polycystic kidney disease.

29. The method of claim 27, wherein the compound is a RPF polypeptide, a nucleic acid encoding a RPF polypeptide, or a nucleic acid that modulates the expression of a nucleic acid that encodes a RPF polypeptide.

30. The method of claim 27, wherein said subject is a human or rodent.

31. The method of claim 27, wherein said compound increases RPF expression or activity.

32. The method of claim 27, wherein said compound decrease RPF expression or activity.

33. An isolated nucleic acid comprising a nucleic acid sequence selected from the group consisting of a SEQ ID NO. 1 and SEQ ID NO. 3 nucleic acid, or its complement.

34. A vector comprising the nucleic acid of claim 33.

35. A cell comprising the vector of claim 34.

36. A pharmaceutical composition comprising the nucleic acid of claim 33.

37. A polypeptide encoded by the nucleic acid of claim 33.

38. An isolated polypeptide comprising the amino acid sequence of SEQ ID NO.2.

39. A kit which detects two or more of the nucleic acid sequences selected from the group consisting of RPF 1-104.

40. An array which detects one or more of the nucleic acids selected from the group consisting of RPF 1-104.

41. A plurality of nucleic acids comprising one or more of the nucleic acids selected from the group consisting of RPF 1-104.
